(19) United States
(12) Patent Application Publication
(10) Pub. No.: US 2024/0148327 A1
(43) Pub. Date: May 9, 2024

(54) DETECTING DEVICE

(71) Applicant: EARFREDO CO., LTD., Hyogo (JP)

(72) Inventor: Shuichi TASAKA, Takarazuka-shi (JP)

(73) Assignee: EARFREDO CO., LTD., Hyogo (JP)

(21) Appl. No.: 18/052,543

(22) Filed: Nov. 3, 2022

(51) Int. Cl. A61B 5/00

(52) U.S. Cl. CPC A61B 5/6817; A61B 2562/164

(57) ABSTRACT

A detecting device includes: a casing at least partly having a curved portion; a sensor unit that is long, has a film shape, and is disposed along a circumferential direction on a first side facing a living body out of an outer side and an inner side of the curved portion; a first elastic member disposed on the first side to be contactable to the living body, and disposed to contact the sensor unit to be able to transmit displacement received from the living body to the sensor unit; and a second elastic member disposed on the first side and supporting the sensor unit in a direction from a second side opposite to the first side toward the first side so that the sensor unit is displaceable based on the displacement received from the living body. The sensor unit outputs an electric signal based on displacement of the sensor unit.

### BACKGROUND OF THE INVENTION

### Field of the Invention

The present disclosure relates to a detecting device. Particularly, the present disclosure relates to a detecting device that is attached to a living body and used for detecting biological information.

### DESCRIPTION OF THE BACKGROUND ART

Japanese Patent Laying-Open No. 2019-55087 (hereinafter referred to as “Patent Document 1”) describes a “biological analysis device”. The biological analysis device includes an optical sensor. The optical sensor includes a light emitting element that emits light toward a living body and a light receiving element that receives light coming from the living body. Biological information can be measured from the detection signal supplied from the optical sensor.

The biological analysis device described in Patent Document 1 uses an LED or the like as the light emitting element, and thus, has a problem of an increase in power consumption. The light emitting element and the light receiving element need to be arranged in an appropriate positional relationship, so that the size of the device increases. Therefore, it is inconvenient to attach this biological analysis device to a living body.

In order to extract information from a living body, a piezoelectric system is also conceivable besides an optical system. In order to achieve a piezoelectric system, it is conceivable to use a film-shaped piezoelectric sensor, but in a detecting device attached to a living body, how to hold the piezoelectric sensor in a state of being capable of acquiring information from the living body is a problem.

In view of this, the present disclosure desirably provides a detecting device capable of stably holding a piezoelectric sensor in a state of being capable of acquiring information from a living body.

### SUMMARY OF THE INVENTION

A detecting device according to the present disclosure includes: a casing that at least partly has a curved portion; a sensor unit that is long and that has a film shape, the sensor unit being disposed along a circumferential direction on a first side which is a side facing a living body out of an outer side and an inner side of the curved portion; a first elastic member disposed on the first side of the curved portion so as to be contactable to the living body, the first elastic member being disposed in contact with the sensor unit so as to be able to transmit displacement received from the living body to the sensor unit; and a second elastic member that supports the sensor unit in a direction from a second side which is a side opposite to the first side toward the first side so that the sensor unit is displaceable on the basis of the displacement received from the living body, the second elastic member being disposed on the first side of the curved portion. The sensor unit outputs an electric signal on the basis of displacement of the sensor unit.

According to the present disclosure, both the first elastic member and the second elastic member are disposed on the first side of the curved portion of the casing, whereby a piezoelectric sensor can be stably held in a state of being capable of acquiring information from the living body.

The foregoing and other objects, features, aspects and advantages of the present disclosure will become more apparent from the following detailed description of the present disclosure when taken in conjunction with the accompanying drawings.

### BRIEF DESCRIPTION OF THE DRAWINGS

FIG. 1 is a perspective view of a detecting device according to a first embodiment of the present disclosure.

FIG. 2 is an exploded view of the detecting device according to the first embodiment of the present disclosure.

FIG. 3 is a side view of the detecting device according to the first embodiment of the present disclosure.

FIG. 4 is an explanatory diagram of a structure of a human ear.

FIG. 5 is a plan view of a first elastic member included in the detecting device according to the first embodiment of the present disclosure.

FIG. 6 is a cross-sectional view taken along line VI-VI in FIG. 5.

FIG. 7 is a cross-sectional view taken along line VII-VII in FIG. 3.

FIG. 8 is a plan view of a sensor unit included in the detecting device according to the first embodiment of the present disclosure.

FIG. 9 is a perspective view of a curved portion that is a part of a casing of the detecting device according to the first embodiment of the present disclosure and that is virtually removed alone from the detecting device.

FIG. 10 is a first explanatory diagram of a first example of operation of attaching the sensor unit to the casing included in the detecting device according to the first embodiment of the present disclosure.

FIG. 11 is a second explanatory diagram of the first example of operation of attaching the sensor unit to the casing included in the detecting device according to the first embodiment of the present disclosure.

FIG. 12 is a first explanatory diagram of a second example of operation of attaching the sensor unit to the casing included in the detecting device according to the first embodiment of the present disclosure.

FIG. 13 is a second explanatory diagram of the second example of operation of attaching the sensor unit to the casing included in the detecting device according to the first embodiment of the present disclosure.

FIG. 14 is a perspective view of a curved portion that is a part of a casing of a detecting device according to a first modification of the first embodiment of the present disclosure and that is virtually removed alone from the detecting device.

FIG. 15 is a perspective view of a curved portion that is a part of a casing of a detecting device according to a second modification of the first embodiment of the present disclosure and that is virtually removed alone from the detecting device.

FIG. 16 is a perspective view of a curved portion that is a part of a casing of a detecting device according to a third modification of the first embodiment of the present disclosure and that is virtually removed alone from the detecting device.

FIG. 17 is a perspective view of a second elastic member and a curved portion that is a part of a casing of a detecting device according to a fourth modification of the first embodiment of the present disclosure, the second elastic member and the curved portion being virtually removed alone from the detecting device.

FIG. 18 is a perspective view of the second elastic member that is included in the detecting device according to the fourth modification of the first embodiment of the present disclosure and that is virtually removed alone from the detecting device.

FIG. 19 is a perspective view of a detecting device according to a second embodiment of the present disclosure.

FIG. 20 is an exploded view of the detecting device according to the second embodiment of the present disclosure.

FIG. 21 is an explanatory diagram of a positional relationship when the detecting device according to the second embodiment of the present disclosure is worn on a human ear.

FIG. 22 is an explanatory diagram of a state when the detecting device according to the second embodiment of the present disclosure is worn on a human ear.

FIG. 23 is a first explanatory diagram of a force that acts when the detecting device according to the second embodiment of the present disclosure is worn on a human ear.

FIG. 24 is a second explanatory diagram of a force that acts when the detecting device according to the second embodiment of the present disclosure is worn on a human ear.

FIG. 25 is a plan view of the detecting device according to the second embodiment of the present disclosure.

FIG. 26 is a third explanatory diagram of a force that acts when the detecting device according to the second embodiment of the present disclosure is worn on a human ear.

FIG. 27 is a side view of the detecting device according to the second embodiment of the present disclosure.

FIG. 28 is a fourth explanatory diagram of a force that acts when the detecting device according to the second embodiment of the present disclosure is worn on a human ear.

### DESCRIPTION OF THE PREFERRED EMBODIMENTS

### First Embodiment

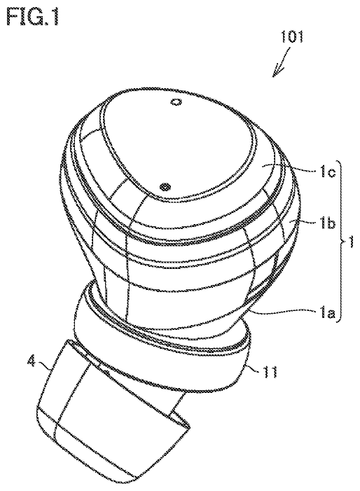

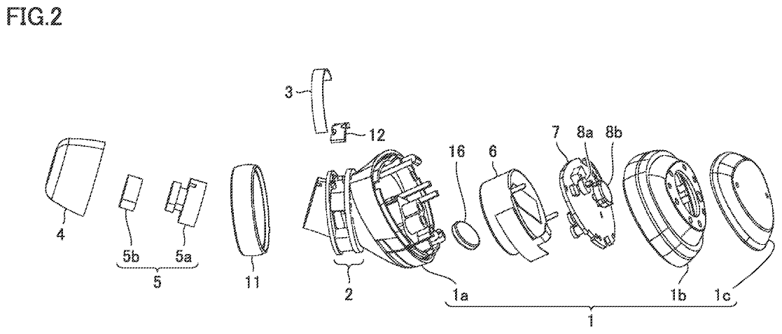

A detecting device according to a first embodiment of the present disclosure will be described with reference to FIGS. 1 to 9. FIG. 1 illustrates a detecting device 101 according to the present embodiment. FIG. 2 is an exploded view of detecting device 101.

Detecting device 101 according to the present embodiment includes a casing 1, a sensor unit 3, a first elastic member 11, and a second elastic member 12. Casing 1 includes a portion 1a, a portion 1b, and a portion 1c. At least a part of casing 1 is a curved portion 2. Curved portion 2 is curved, and thus, has an outer side and an inner side in a conceptual sense. In the example illustrated here, curved portion 2 has a cylindrical shape. As described above, curved portion 2 may have an annular shape obtained by the curved shape being contiguous. Sensor unit 3 is disposed along the circumferential direction on a first side which is the side facing the living body out of the outer side and the inner side of curved portion 2. In the example shown here, the “first side” is the outer side. The sensor unit 3 is long and has a film shape. First elastic member 11 is disposed on the first side, that is, on the outer side of curved portion 2 so as to be able to come into contact with the living body. First elastic member 11 is disposed in contact with sensor unit 3 so as to be able to transmit displacement received from the living body to sensor unit 3. Here, an example in which curved portion 2 has a cylindrical shape is described, but this is merely an example, and curved portion 2 is not necessarily a complete cylindrical shape. In addition, first elastic member 11 has an annular shape, but this is merely an example, and first elastic member 11 is not limited to an annular shape.

Second elastic member 12 supports sensor unit 3 in a direction from a second side that is the inner side out of the outer side and the inner side of curved portion 2, that is, the side opposite to the first side, toward the first side so that sensor unit 3 can be displaced on the basis of the displacement received from the living body. Second elastic member 12 is disposed on the first side, that is, on the outer side of curved portion 2. Sensor unit 3 outputs an electric signal on the basis of the displacement of sensor unit 3.

Casing 1 accommodates a speaker unit 5, a magnet 16, a battery 6, and a substrate 7. Speaker unit 5 includes a driver 5a and a housing 5b. Components 8a and 8b are mounted on the surface of substrate 7.

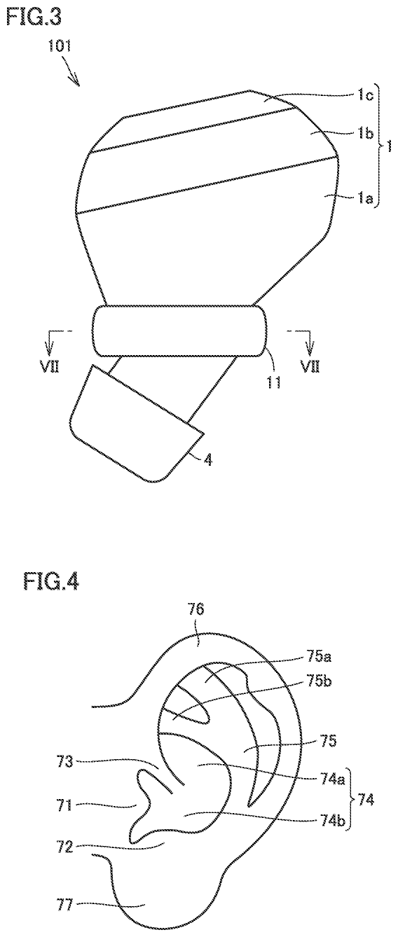

FIG. 3 is a side view of detecting device 101. First elastic member 11 is disposed so as to surround the outer periphery of the intermediate portion of casing 1. Detecting device 101 includes an earpiece 4.

The structure of a human ear is illustrated in FIG. 4. The human ear includes a tragus 71, an antitragus 72, a crus of helix 73, a concha auriculae 74, an antihelix 75, a helix 76, and an earlobe 77. The concha auriculae 74 includes a cymba conchae 74a and a cavum conchae 74b. The antihelix 75 includes an upper leg 75a and a lower leg 75b.

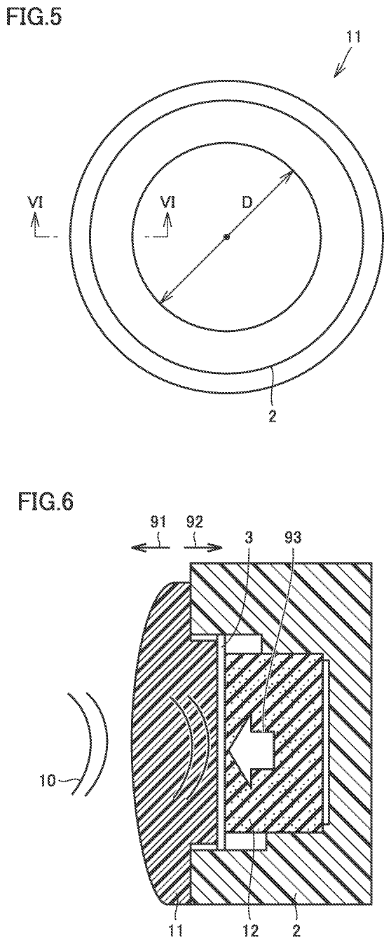

Detecting device 101 is worn on a human ear. When detecting device 101 is worn, earpiece 4 is inserted into the ear canal, and first elastic member 11 is brought into contact with tragus 71. FIG. 5 shows first elastic member 11 removed alone. An inner diameter D is, for example, 12.8 mm. FIG. 6 is a cross-sectional view taken along line VI-VI in FIG. 5. An arrow 91 indicates the first side or outer side, and an arrow 92 indicates the second side or inner side.

An artery passes near tragus 71. A pulsation 10 generated in the artery is transmitted to first elastic member 11 through the skin and further transmitted to sensor unit 3. Sensor unit 3 that is supported by second elastic member 12 in the direction of an arrow 93 is displaceable, and thus, is displaced corresponding to received pulsation 10. As a result, sensor unit 3 converts the displacement into an electric signal and outputs the electric signal.

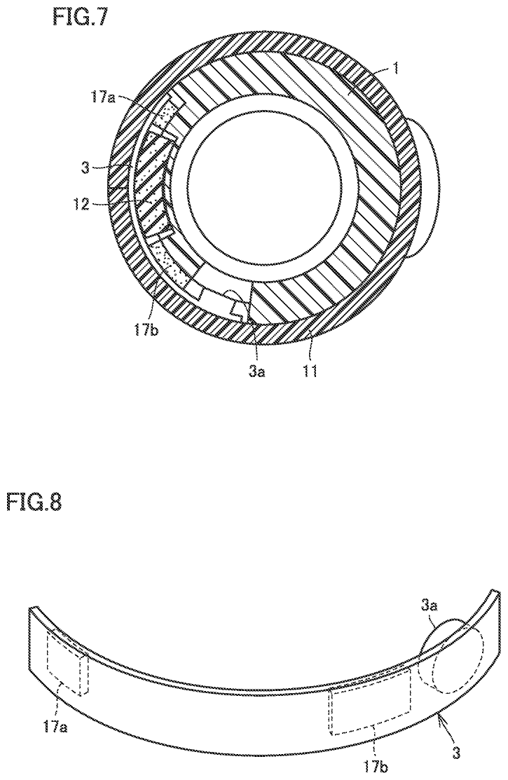

FIG. 7 is a cross-sectional view taken along line VII-VII in FIG. 3. FIG. 8 illustrates sensor unit 3 and double-sided tapes 17a and 17b which are removed. Sensor unit 3 includes a piezoelectric sensor. More specifically, sensor unit 3 includes a piezoelectric film sensor. Sensor unit 3 includes a connection portion 3a in the vicinity of one end. Sensor unit 3 is attached to casing 1 by double-sided tapes 17a and 17b. Double-sided tape 17a is attached to an end of sensor unit 3 distant from connection portion 3a. Double-sided tape 17b is attached to an end of sensor unit 3 closer to connection portion 3b.

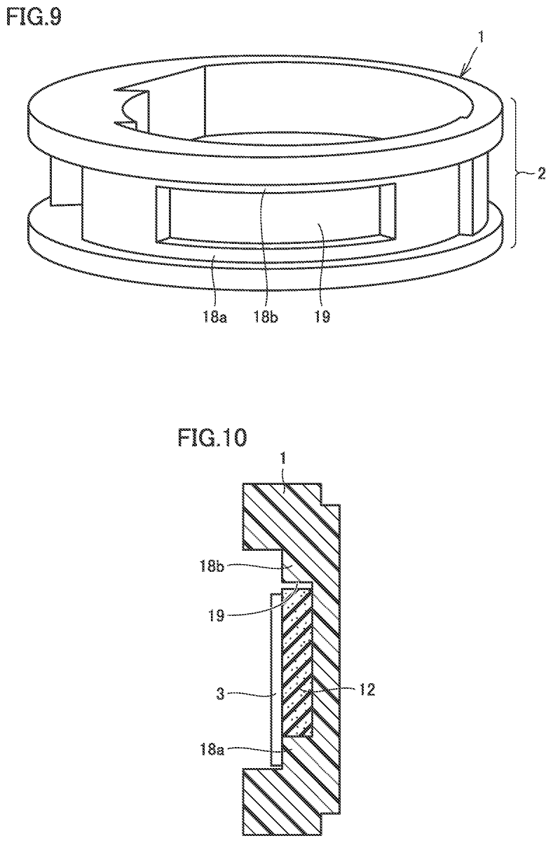

FIG. 9 illustrates curved portion 2 which is a part of casing 1. In practice, casing 1 further extends upward and downward of curved portion 2, and curved portion 2 is not a component that can be removed alone. However, only curved portion 2 is virtually removed here and shown for convenience of description. FIG. 9 illustrates curved portion 2 of casing 1 from which sensor unit 3, first elastic member 11, and second elastic member 12 are removed. Curved portion 2 has a recess 19. Recess 19 is for accommodating second elastic member 12. Guide portions 18a and 18b are provided above and below recess 19.

In detecting device 101 in the present embodiment, sensor unit 3 is supported so as to be displaceable, and thus, can generate an electric signal on the basis of the displacement received from the living body and obtain biological information. In the present embodiment, both first elastic member 11 and second elastic member 12 are disposed on the first side of the curved portion of the casing, whereby sensor unit 3 which is a piezoelectric sensor can be stably held in a state of being capable of acquiring information from the living body.

As described in the present embodiment, both ends of sensor unit 3 in the longitudinal direction are preferably fixed to the surface of curved portion 2 on the first side. The term “both ends” as used herein includes not only a strict end but also a position away from the end to some extent as long as the position is in the vicinity of the end. The positions where double-sided tapes 17a and 17b are attached illustrated in FIGS. 7 and 8 correspond to “both ends”. By applying this configuration, sensor unit 3 can be easily fixed in a proper shape.

As described in the present embodiment, casing 1 preferably includes a guide portion that comes into contact with the intermediate portion of sensor unit 3 from the second side to maintain sensor unit 3 in a shape along the circumference of curved portion 2. In the present embodiment, casing 1 includes guide portions 18a and 18b as the guide portion satisfying such conditions. By applying this configuration, sensor unit 3 can be easily fixed in a proper shape along the circumference of curved portion 2.

When casing 1 includes guide portions 18a and 18b, casing 1 is assembled as illustrated in, for example, FIGS. 10 and 11 when viewed in cross section. Second elastic member 12 and double-sided tapes 17a and 17b have a lot of elasticity and are easily deformed. Therefore, when sensor unit 3 is attached to casing 1, sensor unit 3 is pressed to the right side in the drawing, so that second elastic member 12 is compressed and deformed as illustrated in FIG. 10. That is, sensor unit 3 comes in contact with one of guide portions 18a and 18b, and is not displaced further to the right. In this state, the bonding by double-sided tapes 17a and 17b is completed. When the application of the pressing force is stopped, second elastic member 12 and double-sided tapes 17a and 17b recover from the compressive deformation, and are brought into a state as illustrated in FIG. 11. At this time, sensor unit 3 may not in contact with guide portions 18a and 18b. Sensor unit 3 is bonded to casing 1 by double-sided tapes 17a and 17b.

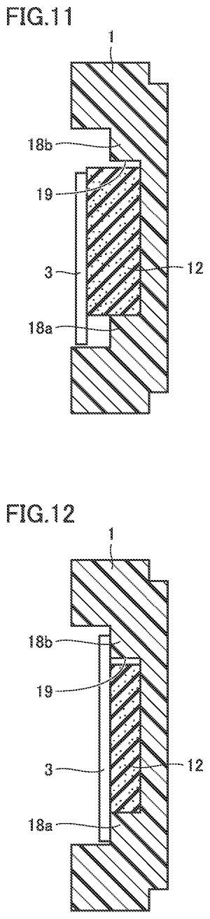

In FIGS. 10 and 11, the width of sensor unit 3 is small, and thus, sensor unit 3 comes in contact with only one of guide portions 18a and 18b, but this is merely an example. For example, sensor unit 3 may have a width large enough to come into contact with both guide portions 18a and 18b. That is, when sensor unit 3 is pressed for bonding, a state as illustrated in FIG. 12 is obtained, and when the application of the pressing force is stopped, a state as illustrated in FIG. 13 is obtained.

Second elastic member 12 is accommodated in recess 19 illustrated in FIG. 9. That is, second elastic member 12 extending in the circumferential direction of curved portion 2 of casing 1 is disposed so as to be sandwiched between guide portions 18a and 18b extending in the circumferential direction of curved portion 2. As described above, it is preferable that guide portions 18a and 18b extend in the circumferential direction of curved portion 2, and second elastic member 12 is disposed along guide portions 18a and 18b. With this configuration, sensor unit 3 can be supported by guide portions 18a and 18b over the entire circumferential length of second elastic member 12.

As a modification of the detecting device in the present embodiment, it is conceivable that curved portion 2 of casing 1 has a different shape. Some modifications of the present embodiment will be described below.

(First Modification)

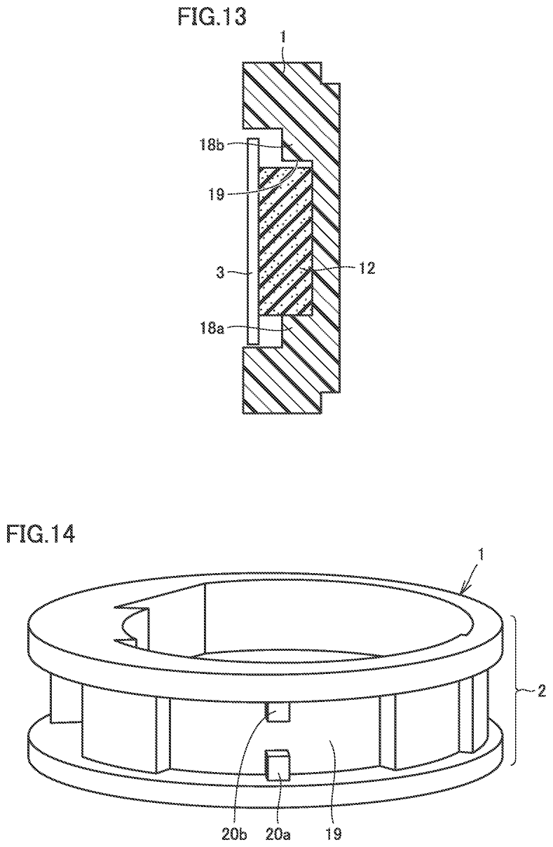

Casing 1 included in a detecting device as a first modification will be described with reference to FIG. 14. FIG. 14 is a perspective view of curved portion 2 of casing 1. In practice, casing 1 may further extend upward and downward of curved portion 2 integrally. However, only curved portion 2 is virtually removed here and shown for convenience of description. In the first modification, curved portion 2 is provided with guide portions 20a and 20b instead of guide portions 18a and 18b. Guide portions 20a and 20b are rectangular parallelepiped protrusions. Guide portions 20a and 20b are provided so as to protrude into recess 19. Guide portions 20a and 20b are provided at the center of recess 19 so as to be vertically symmetrical. Although second elastic member 12 is not illustrated here, second elastic member 12 has a shape that avoids guide portions 20a and 20b. Second elastic member 12 may have an integral shape, or may be divided into two in the horizontal direction across guide portions 20a and 20b.

(Second Modification)

Casing 1 included in a detecting device as a second modification will be described with reference to FIG. 15. In the second modification, curved portion 2 of casing 1 is provided with guide portions 21a and 21b. Guide portions 21a and 21b are arranged at positions shifted in the circumferential direction. Although second elastic member 12 is not illustrated here, second elastic member 12 has a shape that avoids guide portions 21a and 21b. It is preferable that guide portions 21a and 21b are support members arranged at intervals in the circumferential direction of curved portion 2 as described in the second modification, second elastic member 12 has notches, and the support members are disposed inside the notches. By applying this configuration, guide portions 21a and 21b are arranged at different positions in the circumferential direction, and sensor unit 3 can be more uniformly supported.

(Third Modification)

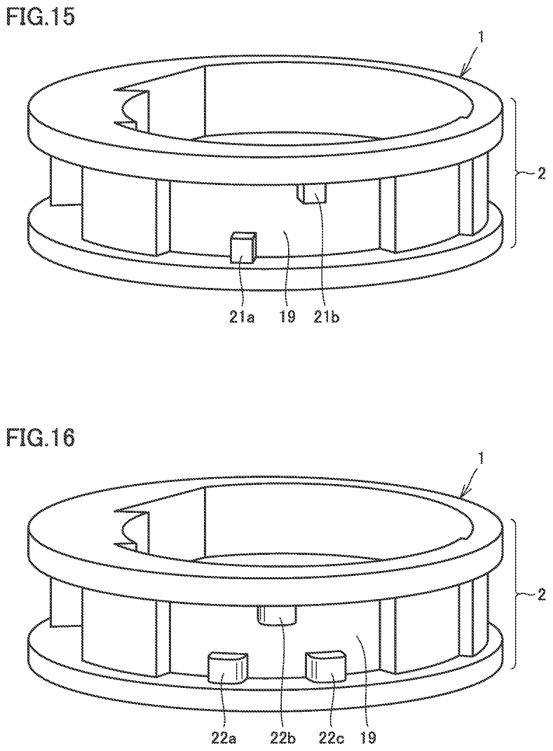

Casing 1 included in a detecting device as a third modification will be described with reference to FIG. 16. In the third modification, curved portion 2 of casing 1 is provided with guide portions 22a, 22b, and 22c. Guide portions 22a, 22b, and 22c are protrusions having a shape of a substantially semicircular column, and are arranged at positions shifted in the circumferential direction. Guide portions 22a and 22c and guide portion 22b are alternately arranged so as to extend from different sides.

(Fourth Modification)

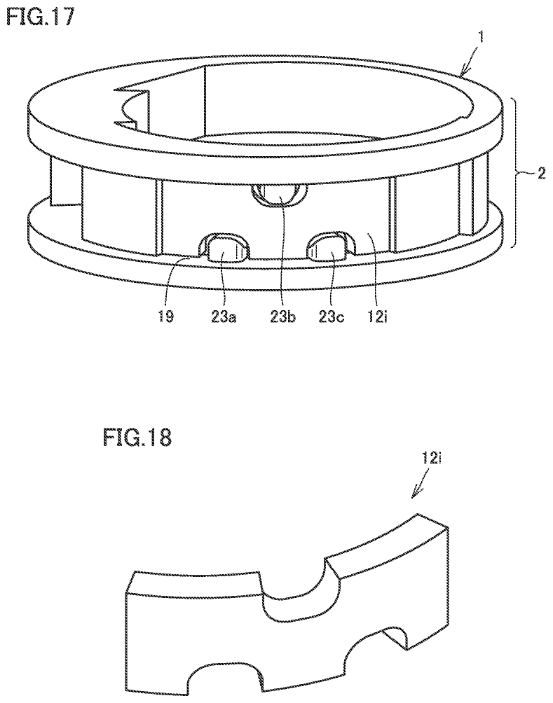

Casing 1 included in a detecting device as a fourth modification will be described with reference to FIG. 17. In the fourth modification, curved portion 2 of casing 1 is provided with guide portions 23a, 23b, and 23c. Guide portions 23a, 23b, and 23c have a substantially semicircular cross section taken along a plane perpendicular to the central axis of curved portion 2, and have a shape in which one end is rounded when viewed from the outer side of curved portion 2 in the radial direction. As illustrated in FIG. 17, a second elastic member 12i is disposed. Second elastic member 12i has a shape that avoids guide portions 23a, 23b, and 23c. FIG. 18 shows second elastic member 12i that is removed alone. Second elastic member 12i has three notches. When second elastic member 12i is disposed in recess 19, guide portions 23a, 23b, and 23c are accommodated in these three notches, respectively.

### Second Embodiment

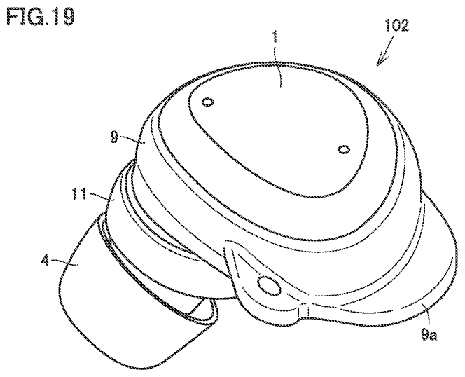

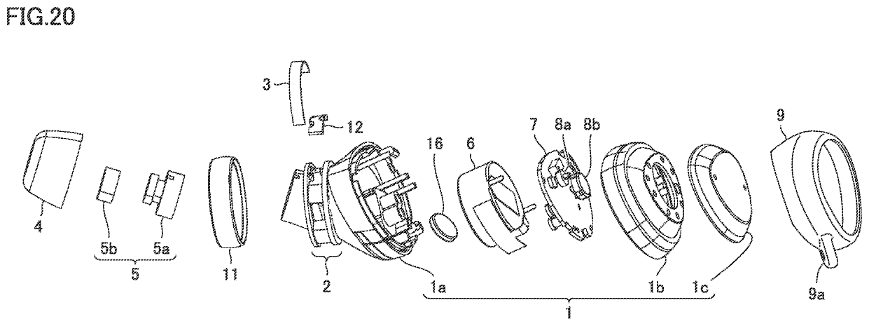

A detecting device according to a second embodiment of the present disclosure will be described with reference to FIGS. 19 to 28. FIG. 19 illustrates a detecting device 102 according to the present embodiment. FIG. 20 is an exploded view of detecting device 102.

Detecting device 102 includes: casing 1 that at least partly has curved portion 2; sensor unit 3 that is long and that has a film shape, sensor unit 3 being disposed along a circumferential direction on a first side which is a side facing a living body out of an outer side and an inner side of curved portion 2; first elastic member 11 disposed on the first side of curved portion 2 so as to be contactable to the living body, first elastic member 11 being disposed in contact with sensor unit 3 so as to be able to transmit displacement received from the living body to sensor unit 3; and second elastic member 12 that supports sensor unit 3 in a direction from a second side which is a side opposite to the first side out of the outer side and the inner side of curved portion 2 so that sensor unit 3 can be displaced on the basis of the displacement received from the living body, second elastic member 12 being disposed on the first side of curved portion 2. Sensor unit 3 outputs an electric signal on the basis of the displacement of sensor unit 3.

In the present embodiment, the living body is a human body. Detecting device 102 is used by being worn on the ear. The first side is the outer side of curved portion 2. Detecting device 102 includes an auxiliary member 9 disposed so as to surround casing 1 at a position shifted from first elastic member 11 in the axial direction of curved portion 2. Auxiliary member 9 includes a contact portion 9a that comes in contact with the living body on a side opposite to a side where first elastic member 11 comes in contact with the living body when viewed from the central axis of curved portion 2. Contact portion 9a is a portion of auxiliary member 9 that protrudes in a plate shape. Auxiliary member 9 comes in contact with the ear, and therefore, is preferably formed of an elastic material so as not to give an uncomfortable feeling when detecting device 102 is worn. Auxiliary member 9 is preferably formed of, for example, rubber. Auxiliary member 9 may be formed of a resin material. In a case where auxiliary member 9 is formed of a resin material, the resin material may be acrylonitrile-butadiene-styrene (ABS) resin, for example. Auxiliary member 9 may be formed integrally with casing 1.

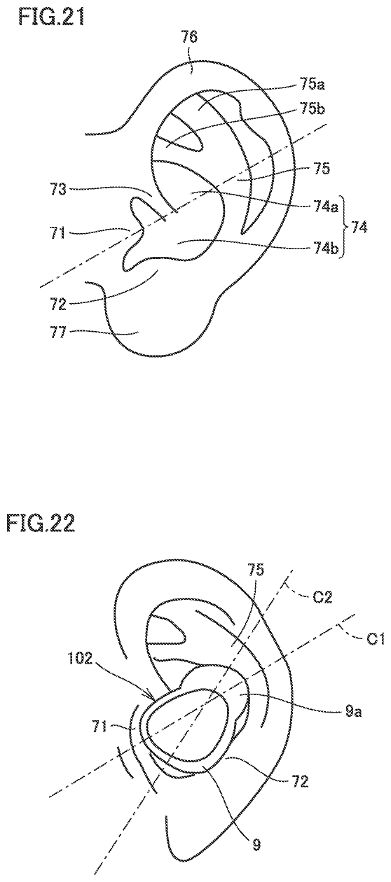

Detecting device 102 is designed based on the following idea. In the human ear, a straight line connecting tragus 71 and antihelix 75 is considered as illustrated by a one-dot chain line in FIG. 21. When worn on a human ear, detecting device 102 is attached so as to extend along this straight line. Detecting device 102 is worn as illustrated in FIG. 22. FIG. 22 illustrates a state in which detecting device 102 is worn on the left ear. In practice, detecting device 102 is pressed perpendicularly against tragus 71 along a straight line C1, and is pressed perpendicularly against antihelix 75 along a straight line C2. Straight line C1 and straight line C2 are not parallel, and the directions thereof are shifted to some extent.

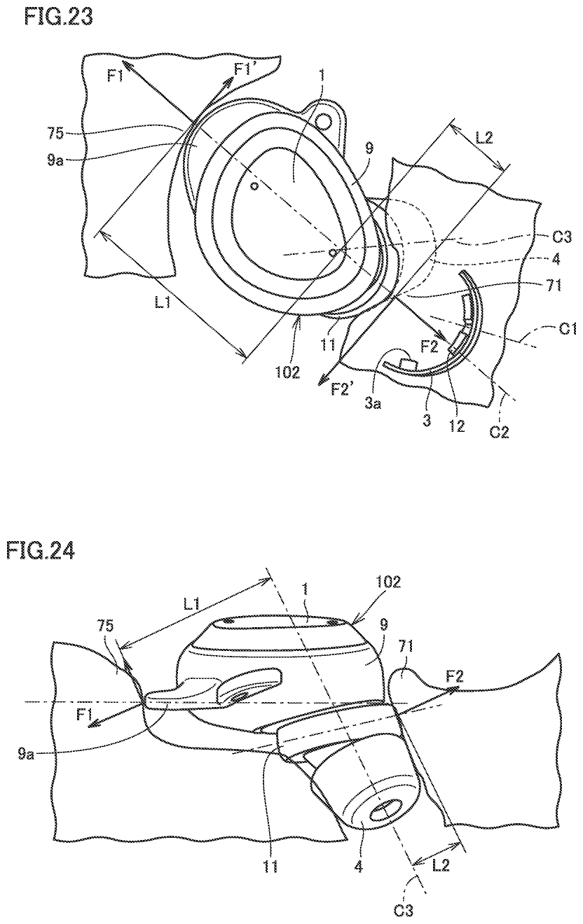

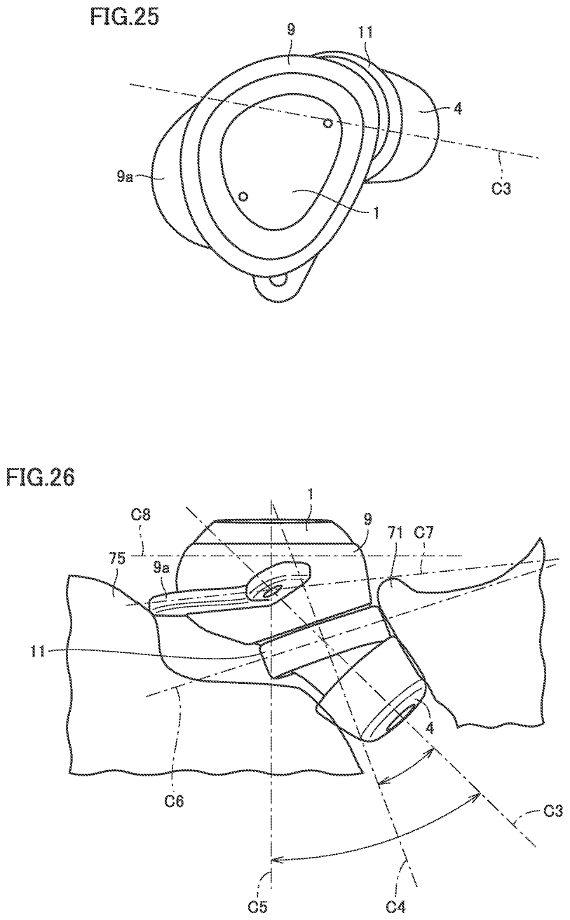

FIGS. 23 to 28 illustrate a more detailed situation where detecting device 102 is worn on the human ear. FIG. 23 is a front view illustrating the state where detecting device 102 is worn on the right ear. In FIG. 23, the right side is the face side, and the left side is the back of the head. In FIG. 23, the posture of sensor unit 3 disposed inside first elastic member 11 is illustrated slightly away from first elastic member 11 to the outside of detecting device 102 as in an exploded view, for convenience of description. Second elastic member 12 is also illustrated together with sensor unit 3, and a straight line passing through the center of second elastic member 12 is straight line C1. The displacement parallel to straight line C1 is detected most easily by sensor unit 3. Contact portion 9a of auxiliary member 9 is pressed against antihelix 75, and first elastic member 11 is pressed against tragus 71. When wearing detecting device 102, a user puts earpiece 4 of detecting device 102 into the ear canal. The center line of earpiece 4 is indicated as straight line C3. In this case, a force F1′ acts on a contact point between contact portion 9a and antihelix 75 with a radius L1. This moment acts as a rubbing motion against tragus 71 with a force FT at a radius L2. L1>L2 is established. Thus, F1′<F2′ is established. Detecting device 102 is put with such a large force, and thus, a part of detecting device 102 can be inserted deeper into the ear canal. The inner diameter of the ear canal is smaller at the back of the ear canal, and thus, detecting device 102 is pressed against the skin with a stronger force. Contact portion 9a is pressed against antihelix 75 with force F1. First elastic member 11 is pressed against tragus 71 with force F2. In this state, force F2 is sufficiently large, so that sensor unit 3 disposed in such a manner that the direction of straight line C1 points to the front can receive the displacement due to the pulsation from the artery at the back of tragus 71. Detecting device 102 may be separately prepared for the right ear and the left ear. FIG. 23 illustrates detecting device 102 for the right ear. Detecting device 102 for the left ear may have a shape symmetrical to that of detecting device 102 for the right ear. Detecting device 102 for the left ear is illustrated in FIGS. 24 and 25. When detecting device 102 includes magnet 16 so that detecting device 102 for the left ear and detecting device 102 for the right ear are attracted to each other, this configuration is convenient for the user, and thus is preferable. In addition, detecting device 102 may be configured such that, when put into a charging case, detecting device 102 can be attracted to the charging case with magnet 16. This configuration stabilizes the position of detecting device 102, and thus is preferable.

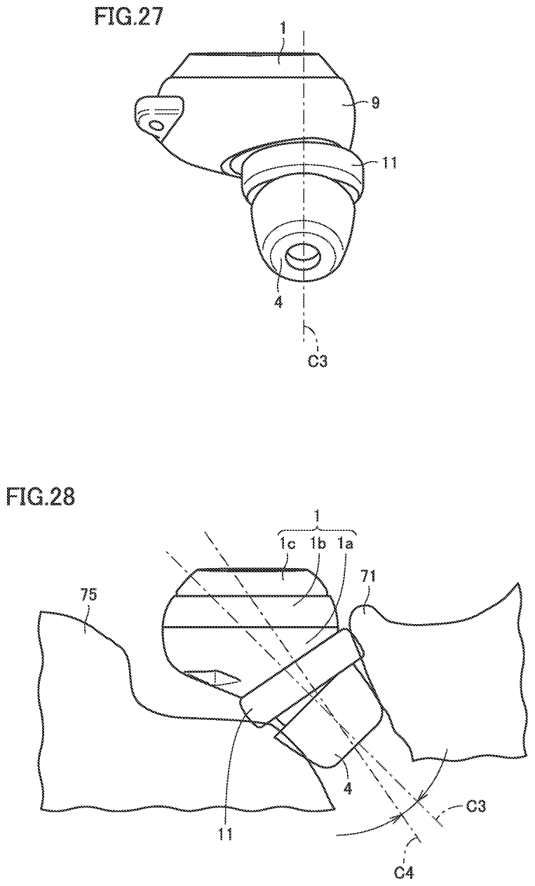

As illustrated in FIGS. 26 and 28, the center line of first elastic member 11 is indicated as a straight line C4. As illustrated in FIG. 26, the center line of portion 1c of casing 1 is indicated as a straight line C5. In the direction viewed in FIG. 26, the diametrical straight line of first elastic member 11 is indicated as a straight line C6. In the direction viewed in FIG. 26, the straight line in a plane direction in which contact portion 9a of auxiliary member 9 extends is indicated as a straight line C7. In the direction viewed in FIG. 26, the straight line in a plane direction in which auxiliary member 9 surrounds casing 1 is indicated as a straight line C8. FIGS. 27 and 28 illustrate a state viewed from a direction different from that in FIG. 26. FIG. 28 illustrates a state in which auxiliary member 9 is removed.

The effects described in the first embodiment can also be obtained in the present embodiment. Furthermore, in the present embodiment, the detecting device includes the auxiliary member, and auxiliary member 9 includes contact portion 9a, whereby contact portion 9a is pressed against antihelix 75, and first elastic member 11 is pressed against tragus 71 by the reaction force. Detecting device 102 is pressed against tragus 71 with a sufficiently large force, and thus, sensor unit 3 can satisfactorily detect the pulsation based on the artery behind tragus 71. Therefore, the biological information can be satisfactorily acquired.

Note that in each of the above embodiments is described an example in which “the first side that is the side facing the living body out of the outer side and the inner side of curved portion 2” is the outer side. In this configuration, the first side is the outer side and the second side is the inner side. In this case, the detecting device is attached such that the living body is positioned outside curved portion 2. That is, the detecting device is attached such that the curved portion of the casing is inserted into a hole-shaped portion such as the ear canal. In this way, the displacement reaching the sensor unit from the outside is detected, whereby the biological information can be obtained.

On the contrary, a detecting device having a configuration in which “the first side that is the side facing the living body out of the outer side and the inner side of curved portion 2” is the inner side is also conceivable. In this configuration, the first side is the inner side and the second side is the outer side. In this case, the detecting device is attached such that the living body is positioned inside curved portion 2. That is, the detecting device may be worn in such a way that, like a ring, the curved portion of the casing may be attached to a finger, for example. In this way, the displacement reaching the sensor unit from the inside is detected, whereby the biological information can be obtained. The location where the detecting device described above is worn is not only a finger but also a wrist, an ankle, a neck, or the like.

Note that the detecting device is not necessarily annular. In the above embodiments is described an example in which the curved portion that is at least a part of the casing of the detecting device is annularly contiguous to form a cylindrical shape. However, this is merely an example, and the curved portion may not be a perfect cylindrical shape. For example, the curved portion of the casing may not have a cylindrical shape, that is, an annular shape, but may be a portion having a shape that forms a part of a cylindrical shape. The curved portion may be annular, but if the curved portion is not annular, the curved portion may have, for example, a C shape or J shape.

For example, the detecting device may be formed as an earphone, a headphone, a hearing aid, or the like of a behind-the-ear design by forming the casing to have a curved portion along the shape of the ear so that the casing incorporating the sensor unit can be hung on the ear. The detecting device may be a bone-conduction earphone, a bone-conduction hearing aid, or the like. The detecting device may be a spectacle frame or the like. The detecting device may have a shape like a head mount used for feeling augmented reality (AR), virtual reality (VR), or the like. If the detecting device as described above is incorporated in a device that comes into contact with the skin of the human body from the outside of the skin and that has a shape like a head mount as described above, the biological information can be obtained by detecting the displacement reaching the sensor unit from the inside.

Although the example in which first elastic member 11 and auxiliary member 9 are different members has been described so far, the first elastic member and the auxiliary member may be integrally formed. With this configuration, the number of components can be reduced. In this case, the first elastic member and the auxiliary member may be integrally formed of the same material, or may be integrally formed of different materials by two-color molding or the like.

Although the present invention has been described and illustrated in detail, it is clearly understood that the same is by way of illustration and example only and is not to be taken by way of limitation, the scope of the present invention being interpreted by the terms of the appended claims.

What is claimed is:

1. A detecting device comprising:
a casing that at least partly has a curved portion;
a sensor unit that is long and that has a film shape, the sensor unit being disposed along a circumferential direction on a first side which is a side facing a living body out of an outer side and an inner side of the curved portion;
a first elastic member disposed on the first side of the curved portion so as to be contactable to the living body, the first elastic member being disposed in contact with the sensor unit so as to be able to transmit displacement received from the living body to the sensor unit; and
a second elastic member that supports the sensor unit in a direction from a second side which is a side opposite to the first side toward the first side so that the sensor unit is displaceable on the basis of the displacement received from the living body, the second elastic member being disposed on the first side of the curved portion, wherein
the sensor unit outputs an electric signal on the basis of displacement of the sensor unit.

2. The detecting device according to claim 1, wherein
both ends of the sensor unit in a longitudinal direction are fixed to a surface of the curved portion on the first side.

3. The detecting device according to claim 1, wherein
the casing includes a guide portion that comes in contact with an intermediate portion of the sensor unit in a direction from the second side toward the first side to maintain the sensor unit in a shape along a circumference of the curved portion.

4. The detecting device according to claim 3, wherein
the guide portion extends in the circumferential direction of the curved portion, and the second elastic member is disposed along the guide portion.

5. The detecting device according to claim 3, wherein
the guide portion is a support member discontinuously disposed in the circumferential direction of the curved portion, the second elastic member has a notch, and the support member is disposed inside the notch.

6. The detecting device according to claim 1, wherein
the living body is a human body,
the detecting device is used by being worn on an ear,
the first side is the outer side of the curved portion, and
the detecting device includes an auxiliary member that is disposed to surround the casing at a position shifted from the first elastic member in an axial direction of the curved portion,
the auxiliary member includes a contact portion that comes in contact with the living body on a side opposite to a side where the first elastic member comes in contact with the living body as viewed in a central axis of the curved portion.

7. The detecting device according to claim 2, wherein
the casing includes a guide portion that comes in contact with an intermediate portion of the sensor unit in a direction from the second side toward the first side to maintain the sensor unit in a shape along a circumference of the curved portion.

8. The detecting device according to claim 2, wherein
the living body is a human body,
the detecting device is used by being worn on an ear,
the first side is the outer side of the curved portion, and
the detecting device includes an auxiliary member that is disposed to surround the casing at a position shifted from the first elastic member in an axial direction of the curved portion,
the auxiliary member includes a contact portion that comes in contact with the living body on a side opposite to a side where the first elastic member comes in contact with the living body as viewed in a central axis of the curved portion.

9. The detecting device according to claim 3, wherein
the living body is a human body,
the detecting device is used by being worn on an ear,
the first side is the outer side of the curved portion, and
the detecting device includes an auxiliary member that is disposed to surround the casing at a position shifted from the first elastic member in an axial direction of the curved portion,
the auxiliary member includes a contact portion that comes in contact with the living body on a side opposite to a side where the first elastic member comes in contact with the living body as viewed in a central axis of the curved portion.

10. The detecting device according to claim 4, wherein
the living body is a human body,
the detecting device is used by being worn on an ear,
the first side is the outer side of the curved portion, and
the detecting device includes an auxiliary member that is disposed to surround the casing at a position shifted from the first elastic member in an axial direction of the curved portion,
the auxiliary member includes a contact portion that comes in contact with the living body on a side opposite to a side where the first elastic member comes in contact with the living body as viewed in a central axis of the curved portion.

11. The detecting device according to claim 5, wherein
the living body is a human body,
the detecting device is used by being worn on an ear,
the first side is the outer side of the curved portion, and
the detecting device includes an auxiliary member that is disposed to surround the casing at a position shifted from the first elastic member in an axial direction of the curved portion,
the auxiliary member includes a contact portion that comes in contact with the living body on a side opposite to a side where the first elastic member comes in contact with the living body as viewed in a central axis of the curved portion.

12. The detecting device according to claim 7, wherein
the guide portion extends in the circumferential direction of the curved portion, and the second elastic member is disposed along the guide portion.

13. The detecting device according to claim 7, wherein
the guide portion is a support member discontinuously disposed in the circumferential direction of the curved portion, the second elastic member has a notch, and the support member is disposed inside the notch.

14. The detecting device according to claim 7, wherein
the living body is a human body,
the detecting device is used by being worn on an ear,
the first side is the outer side of the curved portion, and
the detecting device includes an auxiliary member that is disposed to surround the casing at a position shifted from the first elastic member in an axial direction of the curved portion,
the auxiliary member includes a contact portion that comes in contact with the living body on a side opposite to a side where the first elastic member comes in contact with the living body as viewed in a central axis of the curved portion.

15. The detecting device according to claim 12, wherein
the living body is a human body,
the detecting device is used by being worn on an ear,
the first side is the outer side of the curved portion, and
the detecting device includes an auxiliary member that is disposed to surround the casing at a position shifted from the first elastic member in an axial direction of the curved portion,
the auxiliary member includes a contact portion that comes in contact with the living body on a side opposite to a side where the first elastic member comes in contact with the living body as viewed in a central axis of the curved portion.

16. The detecting device according to claim 13, wherein
the living body is a human body,
the detecting device is used by being worn on an ear,
the first side is the outer side of the curved portion, and
the detecting device includes an auxiliary member that is disposed to surround the casing at a position shifted from the first elastic member in an axial direction of the curved portion,
the auxiliary member includes a contact portion that comes in contact with the living body on a side opposite to a side where the first elastic member comes in contact with the living body as viewed in a central axis of the curved portion.
